(19) United States
(12) Patent Application Publication
(10) Pub. No.: US 2022/0062580 A1
(43) Pub. Date: Mar. 3, 2022

(54) MULTIMODAL PLATFORM FOR ENGINEERING BRAIN STATES

(71) Applicant: X Development LLC, Mountain View, CA (US)

(72) Inventors: Vladimir Miskovic, Binghamton, NY (US); Matthew Dixon Eisaman, Port Jefferson, NY (US); Sarah Ann Laszlo, Mountain View, CA (US); Thomas Peter Hunt, Oakland, CA (US)

(21) Appl. No.: 17/003,620

(22) Filed: Aug. 26, 2020

(51) Int. Cl. A61M 21/02; G16H 20/30; G16H 50/30; G16H 40/67

(52) U.S. Cl. CPC A61M 21/02; G16H 20/30; A61M 2021/0038; G16H 40/67; G16H 50/30

(57) ABSTRACT

A method including identifying an activity pattern of a subject's brain, determining, based on the identified activity pattern of the subject's brain and a target parameter, a set of stimulation parameters, generating, by two or more emitters and based on the set of stimulation parameters, a composite stimulation pattern at a portion of the subject's brain, wherein each of the two or more emitters generates a stimulation pattern using a different modality, measuring, by one or more sensors, a response from the portion of the subject's brain in response to the composite stimulation pattern; and dynamically adjusting, for each emitter and based on the measured response from the portion of the subject's brain, a set of stimulation parameters.

### FIELD

This specification relates to a technological platform for engineering brain states.

### BACKGROUND

Stimulation of the brain in humans is typically performed using a single mode of stimulation and using an open loop system.

### SUMMARY

Brain stimulation is used to treat movement disorders such as Parkinson's disease, tremor, and dystonia, as well as affective disorders such as depression, anxiety, auditory hallucinations, and obsessive-compulsive disorder. Also, there is growing evidence that stimulation can improve memory or modulate attention and mindfulness. Additional therapeutic applications include rehabilitation and pain management.

The methods described here perform non-invasive stimulation of brain networks in real-time and adjust the stimulation based on brain activity patterns. In particular, the methods allow for stimulation that influences the state of a subject's brain activity patterns through multiple, different modes of stimulation. For example, the stimulation can match the natural activity patterns and the complexity of such patterns of a subject's brain. The simultaneous application of these different modes of stimulation provide a flexible platform for engineering brain states that is non-invasive, safe, and reversible.

Machine-learning models can analyze a measured response to transcranial stimulation and generate stimulation parameters. For example, brain activity and function measurements can be used with statistical and/or machine learning models to determine a current brain state, to analyze the response of the subject's brain to the stimulation, and to determine future stimulation parameters. In some cases, the models can be applied to the method to quantify the effectiveness of a particular set of stimulation parameters. The methods can use additional biomarker inputs to determine the stimulation parameters or classify feedback. For example, the methods can use vital signs of the subject or verbal feedback from the subject as additional input to the model to improve the accuracy of the model and to personalize the models to the subject.

Systems for implementing the methods can be embodied in various form factors. In some implementations, the system includes a brain stimulation headset or helmet. In other implementations, the system includes a set of headphones or goggles. The system can include additional components, such as a power system, that are housed separately. For example, the power system for a stimulation headset can be placed in a waist pack.

One innovative aspect of the subject matter described in this specification can be embodied in a method that includes identifying an activity pattern of a subject's brain, determining, based on the identified activity pattern of the subject's brain and a target parameter, a set of stimulation parameters, generating, by two or more emitters and based on the set of stimulation parameters, a composite stimulation pattern at a portion of the subject's brain, wherein each of the two or more emitters generates a stimulation pattern using a different modality, measuring, by one or more sensors, a response from the portion of the subject's brain in response to the composite stimulation pattern, and dynamically adjusting, for each emitter and based on the measured response from the portion of the subject's brain, a set of stimulation parameters.

In some implementations, the target parameter is a selected set of one or more physiological measurements of the subject.

In some implementations, the target parameter is determined based on the subject's feedback.

In some implementations, the different modalities are selected from among ultrasound, pulsed light, or immersive virtual reality.

In some implementations, generating, by two or more emitters and based on the set of stimulation parameters, a composite stimulation pattern at a portion of the subject's brain includes generating, by a first emitter that generates a first stimulation pattern using ultrasound, and generating, by a second emitter that generates a second stimulation pattern using pulsed light. In some implementations, the method further includes generating, by an immersive virtual reality system, based on the set of stimulation parameters, and for presentation to the subject, a visual representation of a scene, and displaying, to the subject, the visual representation of the scene.

In some implementations, dynamically adjusting, for each emitter and based on the measured response from the portion of the subject's brain, a set of stimulation parameters comprises using machine learning or artificial intelligence techniques to generate one or more adjusted stimulation parameters.

In some implementations, the method includes controlling, based on the dynamically adjusted set of stimulation parameters, a set of one or more zone plates.

The details of one or more implementations are set forth in the accompanying drawings and the description, below. Other potential features and advantages of the disclosure will be apparent from the description and drawings, and from the claims.

### BRIEF DESCRIPTION OF THE DRAWINGS

FIG. 1 is a diagram of an example configuration of a multimodal brain stimulation system.

FIG. 2 is a diagram of an example machine learning process for multimodal brain stimulation.

FIG. 3 is a flow chart of an example process of multimodal brain stimulation.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

Non-invasive stimulation of particular regions of a brain, including large-scale brain networks—various sets of synchronized brain areas linked together by brain function—can be used to treat neurological disorders, such as anxiety disorders, trauma and stressor-related disorders, panic disorders, and mood disorders. The methods can also be applied to stimulate peripheral nerves, such as the vagus nerve. Additionally, there has been growing evidence of the positive effects of stimulation of large-scale brain networks on a subject's memory or attention. In general, conventional stimulation of brain networks is not automatically tailored for particular subjects and their needs, and does not take into account brain activity that occurs in response to the stimulation. These methods are typically limited to using a single mode of stimulation, and thus unable to take advantage of the additive benefits of combining stimulation techniques that provide an effect greater than the sum of its parts.

The described methods and systems perform multimodal stimulation of the brain, allow for stimulation of large-scale brain networks in real-time, and adjust the stimulation parameters, including waveform shape and duty cycle, position and intensity, and visual display parameters based on brain activity patterns. The described systems and methods allow for stimulation through pulsed, focused ultrasound beams, rhythmic neurosensory stimulation, and immersive VR technology. In particular, the system can detect and classify a subject's natural brain activity patterns and determine appropriate stimulation parameters to engineer particular brain states, or patterns of neural activity. Brain activity and function measurements can be used with statistical and/or machine learning models to determine a current brain state, to analyze the response of the subject's brain to the stimulation, and to determine future stimulation parameters. In some implementations, the measurements can be used to map out brain electrical conductivity, connectivity, and functionality to personalize stimulation to a particular subject.

For example, the described methods can include providing stimulation according to a particular pattern to a particular area of a subject's brain, contemporaneously or near-contemporaneously recording brain activity detected by sensors, designing stimulation field patterns based on the detected brain activity plus physiological signals such as heart rate and eye movement, and applying the designed stimulation field patterns.

The described methods and systems can be implemented automatically (e.g., without direct human control). For example, the controller can automatically determine the activity pattern of a particular subject's brain along with complimentary physiological signals to tailor stimulation patterns and detection techniques to the particular subject's brain.

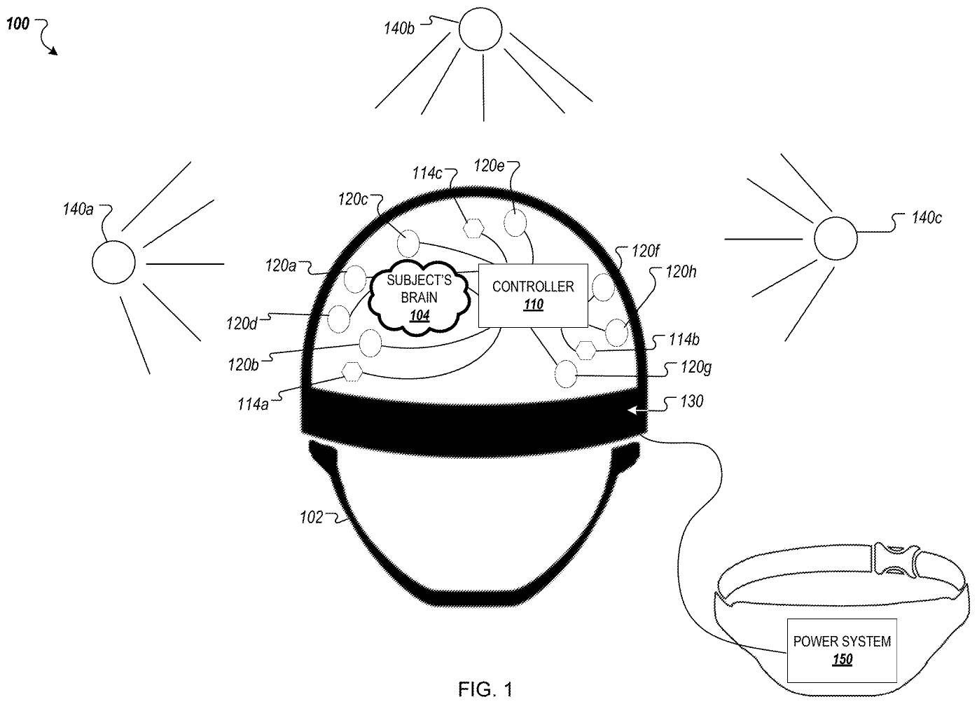

FIG. 1 is a diagram of an example configuration 100 of a multimodal brain stimulation system. For example, system 100 can be used to stimulate one or more target areas of a subject's brain and, based on measured brain activity, system 100 can adjust various parameters of the stimulation of the target area. As a multimodal system, system 100 can be used to simultaneously stimulate a subject's brain using two or more modalities. Typically, brain stimulation systems only provide stimulation through a single mode of stimulation, and are unable to combine different types of stimulation to provide a cumulative effect.

System 100 combines the strengths and limitations of multiple modalities of neurostimulation to create an aggregate, flexible platform for engineering brain states. In this particular example, system 100 uses a multimodal approach that involves triangulation of three specific modalities into one platform, the modalities being: ultrasound, rhythmic neurosensory stimulation, and immersive VR technology. In some implementations, system 100 can use other modalities of neurostimulation, including electrical and magnetic forms of stimulation. System 100's aggregate effect is greater than the sum of its parts, as system 100 allows for different modalities to be tuned differently to achieve effects on a subject 102 ranging from changes in mood to cognitive rest and enhancement to altered, dream-like states of waking consciousness. By combining different modalities of neurostimulation, system 100 allows for exploration of a state space of possible brain states that has not previously been accessible through traditional methods of stimulation. For example, ultrasonic stimulation of neural networks of a subject's brain can replicate some aspects of a brain state, but perceptive effects may be more difficult to achieve; virtual reality systems provide a user with perceptive effects; and rhythmic stimulation through, for example, pulsed light, can induce dream-like effects in a subject and affect brain state through brain-wave entrainment.

The brain states induced by system 100 can provide therapeutic effects. For example, system 100 can be used to treat disorders, such as insomnia, by replicating the brain state that occurs when a subject is in a sleep state to take advantage of synaptic plasticity, the ability of synapses to strengthen or weaken over time in response to increases or decreases in their activity. For example, system 100 can use ultrasonic stimulation through ultrasonic stimulation system 120 to influence the activity patterns of subject's brain 104 to match sleep state activity and use full-field light stimulation through neurosensory stimulation system 140 to place subject's brain 104 in a state that more closely matches a sleep state.

System 100 can also be used to create an altered state of consciousness by using the composite effects of the subsystems 120, 130, and 140 to influence the activity patterns of subject's brain 104.

System 100 includes a controller 110, an ultrasound stimulation system 120, an immersive virtual reality system 130, and a neurosensory stimulation system 140. System 100 provides a high degree of control over stimulation parameters and patterns, allowing stimulation parameters for each modality to be controlled independently. In some implementations, system 100 can simultaneously provide stimulation of a particular modality according to multiple different parameters at multiple different target locations.

Subject 102 is a human subject of transcranial stimulation.

A focal spot, or target area, within subject's brain 104 can be targeted for stimulation. The target area can be, for example, a specific large-scale brain network associated with a particular state of subject's brain 104. In some implementations, the target area can be automatically selected based on detection data. For example, the system 100 can adjust the targeted area within subject's brain 104 based on detected brain activity. In some implementations, the target area can be selected manually based on a target reaction from subject's brain 104 or a target reaction from other body parts of the subject. In some implementations, system 100 can stimulate peripheral nerves in addition to brain regions. For example, system 100 can stimulate peripheral nerves such as the vagus nerve to treat affective disorders such as post-traumatic stress disorder (PTSD), depression, or anxiety through a non-chemical avenue.

System 100 is shown to include sensors 114a, 114b, and 114c (collectively referred to as sensors 114 or sensing system 114). Sensors 114 detect activity of subject's brain 104. Detection can be done using electrical, optical, and/or magnetic techniques, such as EEG, MEG, and MRI, among other types of detection techniques. For example, sensors 114 can include non-invasive sensors such as EEG sensors, MEG sensors, heart rate sensors, and eye movement sensors, among other types of sensors. Sensors 114 can also include temperature sensors, infrared sensors, light sensors, and blood pressure monitors, among other types of sensors. In addition to detecting activity of the subject's brain 104, sensors 114 can collect and/or record the activity data and other data associated with subject 102 and provide the data to controller 110.

Sensors 114 can perform optical detection such that detection does not interfere with the frequencies generated by the stimulation subsystems of system 100. For example, sensors 114 can perform near-infrared spectroscopy (NIRS) or ballistic optical imaging through techniques such as coherence gated imaging, collimation, wavefront propagation, and polarization to determine time of flight of particular photons. Additionally, sensors 114 can collect biometric data associated with subject 102. For example, sensors 114 can detect the heart rate, eye movement, and respiratory rate, among other biometric data of the subject 102.

Ultrasound stimulation system 120 includes transducers or emitters 120a, 120b, 120c, 120d, 120e, 120f, 120g, and 120h (collectively referred to as emitters 120). System 100 is configured to provide stimulation of large-scale brain networks through use of one or more emitters 120. The emitters can provide electrical, magnetic, and/or ultrasound stimulation. The emitters can be, for example, wet electrodes or dry electrodes.

System 100 can stimulate subject's brain 104 using methods such as electrical, magnetic, and ultrasonic stimulation. The configuration of system 100's emitters 120 are dependent on the modality of stimulation. For example, in some implementations in which system 100 uses magnetic stimulation techniques, emitters 120 can be located somewhere other than subject 102's head.

Emitters 120 generate one or more ultrasonic pulsed beams toward a target area within a subject's brain 104. System 100 includes multiple emitters 120, which can generate multiple beams at a focal point, such as a target area within subject's brain 104. Emitters 120 can be powered by direct current or alternating current. Emitters 120 can be identical to each other. In some implementations, emitters 120 can include emitters made of different materials.

In some implementations, sensors 114 can include emitters that emit and detect electrical activity within the subject's brain 104. For example, emitters 120 can include one or more of sensors 114. In some implementations, emitters 120 include each of sensors 114; and the same set of emitters can perform the stimulation and detection of brain activity in response to the stimulation. In some implementations, one subset of emitters may be dedicated to stimulation and another subset dedicated to detection. In some implementations, the stimulation system, i.e., emitters 120, and the detection system, i.e., sensors 114, are electromagnetically or physically shielded and/or separated from each other such that fields from one system do not interfere with fields from the other system. In some implementations, system 100 allows for contemporaneous or near-contemporaneous stimulation and measurement through, for example, the use of high-performance filters that allow for high frequency stimulation at a high amplitude during low noise detection.

Immersive virtual reality system 130 provides subject 102 with a simulated experience. In some implementations, immersive virtual reality systems can be used to treat anxiety disorders. Immersive virtual reality system 130 generates realistic images, sounds and other sensations that simulate a user's physical presence in a virtual environment. Immersive virtual reality system 130 can include visual, audio, and tactile systems that provide stimulation to subject 102. For example, immersive virtual reality system 130 can include a stereoscopic head-mounted display, a stereo sound system, and motion tracking sensors such as gyroscopes, accelerometers, magnetometers, and structured light systems, among other types of tracking sensors. Immersive virtual reality system 130 can include other tracking sensors, including eye tracking sensors. Immersive virtual reality system 130 can provide feedback to subject 102 through systems such as sensory and force feedback. For example, immersive virtual reality system 130 can include a haptic feedback system that provides the experience of touch by applying forces, vibrations, or motions to subject 102. Immersive virtual reality system 130 can include auditory devices such as microphones and/or speakers. Immersive virtual reality system 130 can also be used to induce auditory hallucinogenic effects through sound modulating.

When using immersive virtual reality system 130, subject 102 can interact with the artificial environment. For example, subject 102 can look around, move in, and otherwise interact with features or items within the environment. In some implementations, immersive virtual reality system 130 includes a camera that records subject 102's actual environment and displays the recorded footage to subject 102. For example, the camera can be a forward-facing external camera that records subject 102's actual environment and re-projects the actual environment to subject 102. The external facing camera and the display of immersive virtual reality system 130 provide augmented reality functionality that can modify the actual environment while it is being displayed to subject 102. Immersive virtual reality system 130 can include multiple cameras. For example, immersive virtual reality system 130 include a camera that faces subject 102's back and can project footage of subject 102's back to the subject 102. In some implementations, immersive virtual reality system 130 can induce an out of body experience by projecting, for example, footage of subject 102's environment that subject 102 is not usually able to see.

Neurosensory stimulation system 140 provides subject 102 with stimulation to drive neuronal changes in subject 102. Neurosensory stimulation system 140 provides rhythmic stimulation of a target area of subject 102. In some implementations, neurosensory stimulation system 140 provides rhythmic stimulation through methods including magnetic or electrical stimulation of a particular group of nerves. In this particular example, neurosensory stimulation system 140 includes neurosensory stimulation emitters 140a, 140b, and 140c (collectively referred to as neurosensory stimulation emitters 140 or neurosensory stimulation system 140) that provide stimulation in the form of pulsed light directed to a particular area of subject 102.

Neurosensory stimulation system 140 provides a method of neuromodulation that can be used to drive brain activity. Neurosensory stimulation system 140 can perform forward driving, or “entrainment” of subject's brain 104 to respond to stimulation injected into the brain's activity system. For example, by providing rhythmic stimulation in the form of pulsed light to a target area of subject 102, neurosensory stimulation system 140 can influence subject's brain 104's neural oscillations to follow a frequency of the pulsed light being provided by neurosensory stimulation system 140. Neurosensory stimulation system 140 can also provide, for example, stimulation in the form of uniform light that does not pulse or stable fields of light that are presented within a portion (or the entirety) of, subject 102's field of view, and other types of stimulation to engineer altered perception and dream-like patterns of activity, or states, of subject's brain 104. Neurosensory stimulation system 140 creates and/or alters brain states by subjecting subject's brain 104 to stimulation, such as visible light, with which subject's brain 104 is not familiar.

System 100 is able to target different areas and evoke different responses depending on the spatial precision and type of stimulation that can be achieved by ultrasound stimulation system 120, immersive virtual reality system 130 and neurosensory stimulation generation system 140. For example, ultrasound emissions can provide higher spatial resolution than electrical or magnetic stimulation. System 100 can stimulate different nodes or portions of brain networks using ultrasound emissions as compared to electrical or magnetic emissions.

The composite effects of system 100 can engineer brain states in subject 102 that are not achievable by the subsystems of system 100 individually. For example, system 100 can produce hallucinogenic brain states by combining natural scenes projected through immersive virtual reality system 130 and deep neural network stimulation through ultrasound stimulation system 120 to create perceptual phenomenology without ingesting chemical agents.

In one example, system 100 can be used to create a hallucinogenic effect using immersive virtual reality system 130 to project a street scene to subject 102 in addition to ultrasonic stimulation of a particular neural network of subject's brain 104 provided by ultrasound stimulation system 120 to induce subject 102 to believe they are passing through a street.

In some implementations, system 100 allows contemporaneous or near-contemporaneous detection and stimulation, facilitating a transcranial stimulation system that is able to target large-scale brain networks of subject's brain 104 in real-time and make adjustments to the stimulation based on the detected data. Detection and stimulation may alternate with a period of seconds or less to enable the real-time or near-real-time system. Detection and stimulation signals can be multiplexed. System 100 can also measure phase locking between large-scale brain networks, such that system 100 can apply stimulation to a target area of subject's brain 104 with a known phase delay from a reference signal.

Controller 110 controls and coordinates the various subsystems of system 100. For example, controller 110 allows system 100 to target areas and control stimulation parameters of the different modalities of stimulation available. Controller 110 allows system 100 to apply stimulation through multiple modalities to a target area of subject's brain 104 in-phase with contemporaneous or near-contemporaneous brain signal measurements.

Controller 110 can target multiple, different sizes of spectral areas or different brain regions for different purposes.

Controller 110 includes one or more computer processors that control the operation of various components of system 100, including sensors 114, emitters 120 and components external to system 100, including systems that are integrated with system 100. Controller 110 provides transcranial colored noise stimulation.

Controller 110 generates control signals for the system 100 locally. The one or more computer processors of controller 110 continually and automatically determine control signals for the system 100 without communicating with a remote processing system. For example, controller 110 can receive brain activity feedback data from sensors 114 in response to stimulation from emitters 120 and process the data to determine control signals and generate control signals for emitters 120 to alter or maintain one or more fields generated by emitters 120 within the target area of subject's brain 104.

Controller 110 controls sensors 114 to collect and/or record data associated with subject's brain 104. For example, sensors 114 can collect and/or record data associated with stimulation of subject's brain 104. In some implementations, controller 110 can control sensors 114 to detect the response of subject's brain 104 to stimulation generated by emitters 120. Sensors 114 can also measure brain activity and function through optical, electrical, and magnetic techniques, among other detection techniques.

Controller 110 is communicatively connected to sensors 114. In some implementations, controller 110 is connected to sensors 114 through communications buses with sealed conduits that protect against solid particles and liquid ingress. In some implementations, controller 110 transmits control signals to components of system 100 wirelessly through various wireless communications methods, such as RF, sonic transmission, electromagnetic induction, etc.

Controller 110 can receive feedback from sensors 114. Controller 110 can use the feedback from sensors 114 to adjust subsequent control signals to system 100. The feedback, or subject's brain 104's response to stimulation generated by emitters 120 can have frequencies on the order of tens of Hz and voltages on the order of μV. Subject's brain 104's response to stimulation generated by emitters 120 can be used to dynamically adjust the stimulation, creating a continuous, closed loop system that is customized for subject 102.

Controller 110 can be communicatively connected to sensors other than sensors 114, such as sensors external to the system 100, and uses the data collected by sensors external to the system 100 in addition to the sensors 114 to generate control signals for the system 100. For example, controller 110 can be communicatively connected to biometric sensors, such as heart rate sensors or eye movement sensors, that are external to the system 100.

Controller 110 can accept input other than EEG data from the sensors 114. The input can include sensor data from sensors separate from system 100, such as temperature sensors, light sensors, heart rate sensors, and blood pressure monitors, among other types of sensors. In some implementations, the input can include user input. In some implementations, and subject to safety restrictions, a subject can adjust the operation of the system 100 based on the subject's comfort level. For example, subject 102 can provide direct input to the controller 110 through a user interface. In some implementations, controller 110 receives sensor information regarding the condition of a subject. For example, sensors monitoring the heart rate, respiratory rate, temperature, blood pressure, etc., of a subject can provide this information to controller 110. Controller 110 can use this sensor data to automatically control system 100 to alter or maintain one or more fields generated within the target area of subject's brain 104.

Controller 110 allows for input from a user, such as a healthcare provider or a subject, to guide the stimulation. Rather than being fixed to a specific random noise waveform, controller 110 allows a user to feed in waveforms to control the stimulation to a subject's brain.

Controller 110 uses data collected by sensors 114 and sources separate from system 100 to reconstruct characteristics of brain activity detected in response to stimulation from emitters 120, including the location, amplitude, frequency, and phase of large-scale brain activity. For example, controller 110 can use individual MRI brain structure maps to calculate electric field locations within a particular brain, such as subject's brain 104.

System 100 can operate without feedback in an open loop mode or with feedback in a closed loop mode. In its closed loop mode, system 100 continuously adjusts the applied modality, location, intensity, and other parameters based on feedback such as sensor data including electroencephalogram (EEG) data, eye movement data, heart rate data, and verbal feedback from subject 102 or other physiological signals, among other types of feedback.

Controller 110 controls the selection of which of emitters 120 to activate for a particular stimulation pattern. Controller 110 controls the voltage, frequency, and phase of electric fields generated by emitters 120 to produce a particular stimulation pattern. In some implementations, controller 110 uses time multiplexing to create various stimulation patterns of electric fields using emitters 120. In some implementations, controller 110 turns on various combinations of emitters 120, which may have differing operational parameters (e.g., voltage, frequency, phase) to create various stimulation patterns of electric fields.

Controller 110 selects which of emitters 120 to activate and controls emitters 120 to generate, for example, ultrasonic beams at a target area of subject's brain 104 based on detection data from sensors 114 and stimulation parameters for subject 102. In some implementations, controller 110 selects particular emitters based on the position of the target area. For example, controller 110 can select opposing emitters closest to the target area within subject's brain 104. In some implementations, controller 110 selects particular emitters based on the stimulation to be applied to the target area. For example, controller 110 can select emitters capable of producing a particular intensity or frequency of ultrasonic beam at the target area.

In some implementations, controller 110 operates multiple emitters 120 to generate electrical fields at the target area of subject's brain 104. Controller 110 operates multiple emitters 120 to generate electric fields using direct current or alternating current. Controller 110 can operate multiple emitters 120 to create interfering electric fields that interfere to produce fields of differing frequencies and voltage. For example, controller 110 can operate two opposing emitters 120 (e.g., emitters 120a and 120h) to generate two electric fields having frequencies on the order of kHz that interfere to produce an interfering electric field having a frequency on the order of Hz. Controller 110 can control operational parameters of emitters 120 to generate electric fields that interfere to create an interfering field having a particular beat frequency.

Controller 110 operates neurosensory stimulation emitters 140 to generate pulsed light at a target area of subject 102. In some implementations, the target area is generally within subject 102's field of view. Controller 110 can operate neurosensory stimulation emitters 140 to generate stimulation according to particular steering and operating parameters. Operating parameters can include color, intensity, and duty cycle of light generated. For example, controller 110 can operate neurosensory stimulation emitters 140 to produce a particular wavelength, such as infrared, visible red light, or visible blue light, among other wavelengths of light. Operating parameters can also include size and location at which light should be directed. For example, controller 110 can operate neurosensory stimulation emitters 140 to produce light within subject 102's full field of view. In some implementations, the portion of subject 102's full field of view is correlated with the strength of the effects produced by the stimulation.

System 100 can include one or more zone plates for focusing and steering the stimulation systems, including ultrasound stimulation system 120 and neurosensory stimulation system 140. For example, each of systems 120 and 140 can include a fixed zone plate pattern. Controller 110 can steer and/or focus the stimulation generated by systems 120 and 140 by mechanically actuating and/or bending one or more zone plates. For example, controller 110 can individually control each zone plate or control a number of zone plates in a particular pattern to steer and/or focus the neurosensory stimulation generated by systems 120 and 140. Controller 110 can, for example, tilt a number of zone plates in a pattern to focus pulsed light from neurosensory stimulation system 140 on a specific region on subject 102 or within subject 102's field of view. Controller 110 can change the focus and/or location of the generated stimulation by changing the angle, arrangement, and/or position of various zone plates, among other techniques to control the zone plates. For example, controller 110 can modulate, bend, twist, and/or reconfigure the pattern of zone plates to steer and/or focus ultrasound beams generated by ultrasound stimulation system 120.

Controller 110 can operate various subsystems of system 100 independently and in coordination to create compounding effects that are greater, or different, than can be achieved using a single modality of stimulation. Controller 110 can also operate a single one of ultrasound stimulation system 120, immersive virtual reality system 130, and neurosensory stimulation system 140 to produce stimulation at different locations and/or having different stimulation parameters.

Controller 110 can operate two or more of the ultrasound stimulation system 120, immersive virtual reality system 130, and neurosensory stimulation system 140 to perform second harmonic generation, or frequency doubling, to achieve stronger effects than can be achieved with a single modality of stimulation. Controller 110 can operate the subsystems of system 100 to target harmonic and subharmonic generation. For example, controller 110 can operate ultrasound stimulation system 120 to generate stimulation at a particular frequency and controller 110 can operate neurosensory stimulation system 140 to generate stimulation at a different frequency to generate a harmonic.

In some implementations, controller 110 can perform frequency tagging by modifying the contrast of the frequency at different temporal frequencies such that emergent frequency components, or intermodulation responses, can be observed. For example, controller 110 can tag five different signals to track different emergent frequency components.

Controller 110 can operate two or more of the ultrasound stimulation system 120, immersive virtual reality system 130, and neurosensory stimulation system 140 to generate stimulation having complex modulation frequencies. For example, controller 110 can generate stimulation signals at frequencies such that the combination of the signals results in subtraction of the two signals.

In some implementations, controller 110 is able to generate constructive and/or destructive signals by combining different signals and modalities. For example, controller 110 can pre-sonicate one or more areas of subject's brain 104 and then electrically stimulate the area to achieve a stronger, or different effect than with electrical stimulation alone.

Various subsystems of system 100 can provide cognitive enhancing effects. For example, immersive virtual reality system 130 and/or neurosensory stimulation system 140 can be used to enhance visual function in subject 102 by training subject's brain 104 to shift the peak of neuronal oscillations in the alpha range (e.g., oscillations in the 6-12 Hz range) to a higher frequency. Immersive virtual reality system 130 can, for example, emit virtual reality imagery or light flickering at a particular frequency to shift the peak of subject's brain 104 neuronal oscillations in the alpha range.

In some implementations, controller 110 can communicate with a remote server to receive new control signals. For example, controller 110 can transmit feedback from sensors 114 to the remote server, and the remote server can receive the feedback, process the data, and generate updated control signals for the system 100 and other components.

System 100 can receive input from subject 102 and automatically determine a target area and control emitters 120 to generate stimulation parameters for a particular type of stimulation at the target area. For example, controller 110 can determine, based on collected feedback information from subject's brain 104 in response to stimulation, an area, or large-scale brain network, to target.

System 100 performs activity detection to uniquely tailor stimulation for a particular subject 102. In some implementations, the system 100 can start with a baseline map of brain conductivity and functionality and dynamically adjust stimulation to the target area of subject's brain 104 based on activity feedback detected by sensors 114. In some implementations, system 100 can perform tomography on subject's brain 104 to generate maps, such as maps of large-scale brain activity or electrical properties of the head or brain. For example, the system 100 can produce large-scale brain network maps for subject's brain 104 based on current absorption data measured by sensors 114 that indicate the amount of activity of a particular area of subject's brain 104 in response to a particular stimulus. In some implementations, system 100 can start with provisionally tailored maps that are generally applicable to a subset of subjects 102 having a set of characteristics in common and dynamically adjust stimulation to the target area of subject's brain 104 based on activity feedback detected by sensors 114.

In some implementations, controller 110 can control emitters 120 such that the intensity of the ultrasonic beams generated are lower than are used in therapeutic applications. Controller 110 operates emitters 120 to produce ultrasonic beams that affect the network state that a subject is in. For example, controller 110 can be used to produce ultrasonic beams that induce a focused state, a relaxed state, or a meditation state, among other states, of subject's brain 104. In some implementations, controller 110 can be used to manipulate the state of subject's brain 104 to increase focus and/or creativity and aid in relaxation, among other network states.

In some implementations, controller 110 can be housed separately from other subsystems of system 100. In some implementations, controller 110 and associated power systems can be integrated with other subsystems of system 100 to provide a more compact, comfortable form factor. In some implementations, controller 110 communicates with a remote computing device, such as a server, that trains and updates controller 110's machine learning models. For example, controller 110 can be communicatively connected to a cloud-based computing system.

System 100 includes safety functions that allow a subject to use the system 100 without the supervision of a medical professional. In some implementations, system 100 can be used by a subject for non-clinical applications in settings other than under the supervision of a medical professional.

In some implementations, various subsystems of system 100 have limits on the intensity and frequency, among other parameters, of the stimulation signals generated. For example, pulsed light produced by neurosensory stimulation system 140 can be limited by a maximum frequency. In some implementations, system 100 can be limited based on conditions specific to subject 102. For example, if subject 102 is known to have sensitivity to pulsed light, neurosensory stimulation system 140 can be adapted such that light emitted by neurosensory stimulation system 140 is uniform, and is not pulsed.

In some implementations, system 100 cannot be activated by a subject without the supervision of a medical professional, or cannot be activated by a subject at all. For example, system 100 may require credentials from a medical professional prior to use. In some implementations, only subject 102's doctor can turn on system 100 remotely or at their office.

In some implementations, system 100 can uniquely identify a subject 102, and may only be used by the subject 102. For example, system 100 can be locked to particular subjects and may not be turned on or activated by any other users.

System 100 can limit the range of frequencies and intensities of the stimulation applied through ultrasound stimulation system 120, immersive virtual reality system 130, and neurosensory stimulation system 140 to prevent delivery of harmful patterns of stimulation. For example, system 100 can detect and classify stimulation patterns as seizure-inducing, and prevent delivery of seizure inducing stimulus. In some implementations, system 100 can detect activity patterns in early stages of the activity and preventatively take action. For example, system 100 can detect activity patterns in an early stage of anxiety and preventatively take action to prevent subject's brain 104 from progressing into later stages of anxiety. System 100 can also detect seizure activity patterns using the extracranial activity and biometric data collected by sensors 114, and adjust the stimulation provided by emitters 120 to prevent subject 102 from having a seizure.

In some implementations, system 100 is used for therapeutic purposes. For example, system 100 can be tailored to a subject 102 and used as a brain activity regulation device that detects epileptic activity within the subject's brain 104 and provides prophylactic stimulation.

Controller 110 can use statistical and/or machine learning models which accept sensor data collected by sensors 114 and/or other sensors as inputs. The machine learning models may use any of a variety of models such as decision trees, linear regression models, logistic regression models, neural networks, classifiers, support vector machines, inductive logic programming, ensembles of models (e.g., using techniques such as bagging, boosting, random forests, etc.), genetic algorithms, Bayesian networks, etc., and can be trained using a variety of approaches, such as deep learning, association rules, inductive logic, clustering, maximum entropy classification, learning classification, etc. In some examples, the machine learning models may use supervised learning. In some examples, the machine learning models use unsupervised learning.

Power system 150 provides power to the various subsystems of system 100 and is connected to each of the subsystems. Power system 150 can also generate power, for example, through renewable methods such as solar or mechanical charging, among other techniques.

In this particular example, power system 150 is shown to be separate from the various other subsystems of system 100. Power system 150 is, in this example, an external power source housed within a separate form factor, such as a waistpack connected to the various subsystems of system 100.

In some implementations, system 100 can be used without an external power source. For example, system 100 can include an integrated power source or an internal power source. The integrated power source can be rechargeable and/or replaceable. For example, system 100 can include a replaceable, rechargeable battery pack that provides power to the emitters and sensors and is housed within the same physical device as system 100.

In this particular example, system 100 is housed within a wearable headpiece that can be placed on a subject's head. In some implementations, system 100 can be implemented as a network of individual emitters and sensors that can be placed on the subject's head or a device that holds individual emitters and sensors in fixed positions around the subject's head. In some implementations, system 100 can be implemented as a device tethered in place and is not portable or wearable. For example, system 100 can be implemented as a device to be used in a specific location within a healthcare provider's office.

Individually, each of ultrasound stimulation system 120, immersive virtual reality system 130, and neurosensory stimulation system 140 produce therapeutic and/or neuromodular effects in a patient through neurostimulation. Combined, system 100 can influence a subject's brain states to an extent beyond what is possible using a single one of the stimulation modalities.

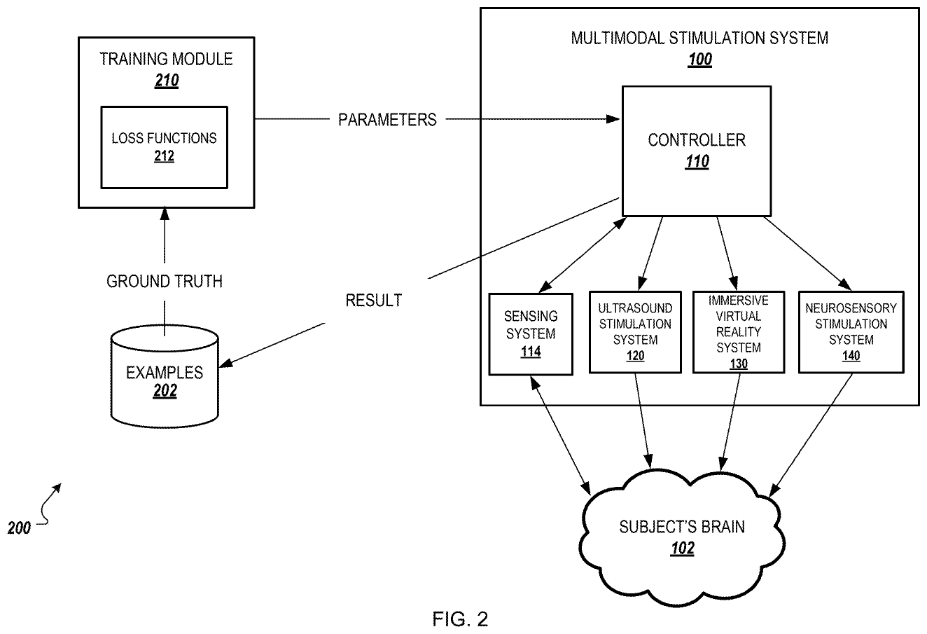

Other form factors for the multimodal stimulation system described in the present application are contemplated. For example, system 100 can be a device that is administered by a healthcare provider to a patient. In some implementations, system 100 can be operated by subject 102 without the supervision of a healthcare provider. For example, system 100 can be provided to patients and can be adjustable by the patient, and in some implementations, can automatically calibrate to the patient and a particular target spot. Automatic targeting and calibration are described with respect to FIG. 2.

System 100 can be implemented as a device worn by subject 102 on their head. In this particular implementation, system 100 is in a comfortable form factor that contacts subject 102 on either side of their head and has the automatic steering and focusing systems as described below. For example, system 100 can be implemented as a pair of headphones.

System 100 can be implemented as a device worn by a subject 102 on their face. In this particular implementation, system 100 is in a comfortable form factor in the shape of eyewear and has the automatic steering and focusing systems as described below. For example, device 420 can be a pair of glasses or goggles.

FIG. 2 is a diagram of an example block diagram of a system 200 for training a multimodal brain stimulation system. For example, system 200 can be used to train multimodal brain stimulation system 100 as described with respect to FIG. 1.

As described above with respect to FIG. 1, system 100 includes a controller 110 that classifies brain activity detected by a sensing system and determines stimulation parameters for a stimulation pattern generation system. For example, controller 110 classifies activity detected by sensors, or sensing system 114, and determines stimulation parameters for emitters, or stimulation pattern generation system 100, including the pattern, frequency, shape, power, and modality. Activity classification can include identifying the location, amplitude, frequency, and phase of large-scale brain activity. Controller 110 can additionally perform functions including quantifying dosages and effectiveness of applied stimulation.

Examples 202 are provided to training module 210 as input to train a machine learning model used by controller 110, such as an activity classification model. Examples 202 can be positive examples (i.e., examples of correctly determined activity classifications) or negative examples (i.e., examples of incorrectly determined activity classifications).

Examples 202 include the ground truth activity classification, or an activity classification defined as the correct classification. Examples 202 include sensor information such as baseline activity patterns or statistical parameters of activity patterns for a particular subject. For example, examples 202 can include tomography data of subject 102's brain 104 generated through activity detection performed by sensors 114 or sensors external to system 100 as described above (e.g., MRIs, EEGs, MEGs, and computed tomography based on the detected data from sensors 114, among other detection techniques). Examples 202 can include statistical parameters of noise patterns of subject 102's brain 104.

In some implementations, the statistical parameters of subject 102's brain 104's noise patterns are closely related to entropic measurements of the patterns. The entropic measurements and noise patterns can be overlapping and capture many of the same properties for the purposes of analyzing the noise patterns.

The ground truth indicates the actual, correct classification of the activity. For example, a ground truth activity classification can be generated and provided to training module 210 as an example 202 by detecting an activity, classifying the activity, and confirming that the activity classification is correct. In some implementations, a human can manually verify the activity classification. The activity classification can be automatically detected and labelled by pulling data from a data storage medium that contains verified activity classifications.

The ground truth activity classification can be correlated with particular inputs of examples 202 such that the inputs are labelled with the ground truth activity classification. With ground truth labels, training module 210 can use examples 202 and the labels to verify model outputs of an activity classifier and continue to train the classifier to improve forward modelling of brain activity through the use of detection data from sensors 114 to predict brain functionality and activity in response to stimulation input.

The sensor information guides the training module 210 to train the classifier to create a morphology correlated map. The training module 210 can associate the morphology of a particular subject's brain 104 with an activity classification to map out brain conductivity and functionality. Inverse modelling of brain activity can be conducted by using measured responses to approximate brain networks that could produce the measured responses. The training module 210 can train the classifier to learn how to map multiple raw sensor inputs to their location within subject's brain 104 (e.g., a location relative to a reference point within subject's brain 104's specific morphology) and activity classification based on a morphology correlated map. Thus, the classifier would not need additional prior knowledge during the testing phase because the classifier is able to map sensor inputs to respective areas within subject's brain 104 and classify activities using the correlated map.

Training module 210 trains an activity classifier to perform activity classification. For example, training module 210 can train a model used by controller 110 to recognize large-scale brain activity based on inputs from sensors within an area of subject's brain 104. Training module 210 refines controller 110's activity classification model using electrical tomography data collected by sensors 114 for a particular subject's brain 104. Training module 210 allows controller 110 to output complex results, such as a detected brain functionality instead of, or in addition to, simple imaging results.

Controller 110 can, for example, adjust brain stimulation parameters based on detected activity patterns. For example, controller 110 may adjust stimulation parameters and patterns based on a property of brains and brain signals known as criticality, where brains can flexibly adapt to changing situations.

In some implementations, controller 110 can apply stimulation patterns that amplify natural brain activity. For example, controller 110 can detect and identify natural activity patterns of brain signals. In one example, an identified activity pattern includes pink noise pattern. Activity patterns can vary, for example, in frequency, power, and/or wavelength.

System 100 performs monitoring of the effects of stimulation. The monitoring can be performed using various methods of measurement. In some implementations, controller 110 can detect and classify psychological states of a subject's brain 104 based on physiological input data. For example, controller 110 can receive input data including eye movements and other biometric measurements. Controller 110 can use eye movement data, for example, to detect cognitive load parameters.

In some implementations, controller 110 can correlate physiological signals with a subject's brain state. For example, controller 110 can calculate an entropic state of subject 102's brain state based on subject 102's eye movement.

In some implementations, controller 110 can receive, for example, verbal output from a subject 102. For example, controller 110 can use techniques such as natural language processing to classify a subject 102's statements. These classifications can be used to determine whether a subject is in a particular psychological state. The system can then use these classifications as feedback to determine stimulation parameters to adjust the stimulation provided to the subject's brain. For example, controller 110 can determine, based on verbal feedback, the emotional content of subject 102's voice and subject 102's brain state. Controller 110 can then determine stimulation parameters to adjust the stimulation provided to subject 102's brain in order to guide subject 102 to a different state or amplify subject 102's current state. For example, controller 110 can perform task-based feedback and classification, where a subject 102 is asked to perform tasks during the stimulation, and subject 102's performance of the task or verbal feedback during their performance of the task is used to determine the subject 102's brain state.

In some implementations, controller 110 can tailor stimulation based on performance metrics such as a measure of the subject's attention or direct subjective feedback, such as how the stimulation makes a subject feel. Feedback can also be derived from the monitoring of peripheral physiological signals, such as, but not limited to, heart rate, heart rate variability, pupil dilation, blink rate, and related measures. This information can be used to model the state of the peripheral nervous system and adjust stimulation parameters accordingly, or even, as a way to quantify the effective dosage of stimulation. For example, stimulation of the cranial nerve (i.e., vagal nerve stimulation) can be quantified by measuring the dilation of a subject's pupil.

Training module 210 trains controller 110 using one or more loss functions 212. For example, training module 210 uses an activity classification loss function 212 to train controller to classify a particular large-scale brain activity. Activity classification loss function 212 can account for variables such as a predicted location, a predicted amplitude, a predicted frequency, and/or a predicted phase of a detected activity.

Training module 210 can train controller 110 manually or the process could be automated. For example, if an existing tomographic representation of subject's brain 104 is available, the system can receive sensor data indicating brain activity in response to a known stimulation pattern to identify the ground truth area within subject's brain 104 at which an activity occurs through automated techniques such as image recognition or identifying tagged locations within the representation. A human can also manually verify the identified areas.

Training module 210 uses the loss function 110 and examples 202 labelled with the ground truth activity classification to train controller 110 to learn where and what is important for the model. Training module 210 allows controller 110 to learn by changing the weights applied to different variables to emphasize or deemphasize the importance of the variable within the model. By changing the weights applied to variables within the model, training module 210 allows the model to learn which types of information (e.g., which sensor inputs, what locations, etc.) should be more heavily weighted to produce a more accurate activity classifier.

Training module 210 uses machine learning techniques to train controller 110, and can include, for example, a neural network that utilizes activity classification loss function 212 to produce parameters used in the activity classifier model. These parameters can be classification parameters that define particular values of a model used by controller 110.

In some implementations, a model used by controller 110 can select a filter to apply to the generated stimulation pattern to stabilize the stimulation being applied to subject 102 when subject 102's brain activity reaches a particular level of complexity.

Controller 110 classifies brain activity based on data collected by sensors 114. Controller 110 performs forward modelling of brain activity and inverse modelling of brain activity, given base, reasonable assumptions regarding the stimulation applied to a target area within subject's brain 104.

Forward modelling allows controller 110 to determine how to propagate waves through subject's brain 104. For example, controller 110 can receive a specified objective (e.g., a network state of subject's brain 104) and design stimulation field patterns to modify brain activity detected by sensors 114. Controller 110 can then control two or more of ultrasound stimulation system 120, immersive virtual reality system 130, and neurosensory stimulation system 140 to apply stimulation to one or more target areas of subject's brain 104 to produce the specified objective network state.

Inverse modelling allows controller 110 to estimate the most likely relationship between the detected activity and the corresponding areas or networks of subject's brain 104. For example, controller 110 can receive brain activity data from sensors 114 and, optionally, physiological data from other sensors, and reconstruct, using an activity classifier model, the location, amplitude, frequency, and phase of the large-scale brain activity. Controller 110 can then dynamically alter the existing activity classifier model and/or tomography representation of subject's brain 104 based on the reconstruction.

Controller 110 can use various types of models, including general models that can be used for all patients and customized models that can be used for particular subsets of patients sharing a set of characteristics, and can dynamically adjust the models based on detected brain activity. For example, the classifier can use a base network for subjects and then tailor the model to each subject. The brain activity can be detected by sensors 114 contemporaneously or near-contemporaneously with the stimulation provided by two or more of ultrasound stimulation system 120, immersive virtual reality system 130, and neurosensory stimulation system 140. In some implementations, the brain activity can be detected through techniques performed by systems external to system 100, such as functional magnetic resonance imaging (fMRI) or diffusion tensor imaging (DTI).

Controller 110 provides stimulation that matches patterns of the natural signals of a subject's brains. Humans shift across brain activity patterns similar to patterns of noise. For example, human brain activity patterns can shift from Brownian noise patterns having low frequencies during sleep, to pink noise patterns as a subject wakes up, to pink and/or white noise patterns as a subject becomes more active. Controller 110 can detect and identify brain activity patterns of a subject 102 and determine, for example, statistical parameters of random noise stimulation patterns that match subject 102's naturally occurring brain activity patterns to amplify the effects of the stimulation. Matching subject 102's naturally occurring brain activity patterns can produce better phase alignment.

Controller 110 can determine, for example, stimulation patterns that match subject 102's naturally occurring Brownian noise patterns, pink noise patterns, and white noise patterns. Controller 110 can then apply white noise patterns to subject 102's brain 104 when subject 102 should be in an active brain state. For example, controller 110 can aid in focus and alertness by matching its patterns of stimulation to subject 102's brain 104's naturally occurring white noise pattern to amplify the effects of stimulation.

In some implementations, controller 110 can apply a signal to the subject's brain to sync the brain to a particular pattern and then transition to a different stimulation pattern. By matching subject 102's brain 104's naturally occurring activity pattern, controller 110 can, in effect, grab the attention of brain 104. Controller 110 can then transition to a different stimulation pattern, leading brain 104 to a different activity pattern.

In addition to matching the statistical activity patterns, controller 110 can also measure the power spectral density of a subject 102's brain state and reproduce the patterns to assist brain 104 in matching the stimulation. For example, controller 110 may want to limit the amount of power provided in the applied stimulation, but the stimulation needs to provide enough power to produce a response. By matching the power spectral density of a brain 104's state, controller 110 can induce maximum self-organized complexity such that brain 104 is guided by later changes in stimulation.

Controller 110 can determine the complexity of a noise pattern occurring in a subject's brain using several different methods of measurement. In some implementations, the complexity of brain signals matches the complexity of the subjective experience a subject is undergoing. For example, brain signals may have limited complexity when a subject is in deep sleep, whereas brain signals may have more complexity when a subject is under the influence of a stimulant.

Controller 110 provides a user with the ability to apply waveforms with various parameters as stimulation to a subject's brain. In some implementations, a user can select a particularly shaped waveform to apply to subject 102's brain 104. For example, a user can apply a triangle wave stimulation pattern to subject 102's brain 104. Different shapes of waveforms can have different effects. Applying a triangle wave stimulation pattern to a subject 102's brain 104 can act as a siren, seizing the attention of brain 104. A user can apply different shapes of wave stimulation patterns including sawtooth, sine, and square waves, among other shapes, to achieve different effects.

The type of stimulation and the areas of a brain that can be stimulated are closely related to, and in some cases, governed by, the modality with which the stimulation is provided. As discussed above, emitters 120 can provide electrical, magnetic, and/or ultrasound stimulation. If, for example, controller 110 applies focused ultrasound stimulation, controller 110 would need to focus and steer a wide bandwidth of the ultrasound beam into a target region.

Ultrasound stimulation provides a wide range and provides resolution on the order of millimeters. With finer resolution, controller 110 can target deep brain structures such as basal ganglia. For example, controller 110 can use ultrasound stimulation to control tremors by detecting the frequency of a tremor, classifying the frequency as a certain color of noise, and applying stimulation to shift the color of noise.

In some implementations, electrical stimulation may provide a coarser resolution than ultrasound stimulation. Electrical stimulation can be applied using, for example, high-definition electrodes that can be used to target regions such as the frontal cortex of a subject's brain to produce cognitive effects.

In addition to controlling the intensity and shape of stimulation signals, controller 110 can control the time scale of signal switching. In some implementations, the switching frequency is lower than that used in focused ultrasound. In some implementations, the switching frequency is adapted based on a subject's natural brain activity pattern frequencies.

Controller 110 can collect response data from subject 102 to quantify dosage provided to subject 102's brain 104. For example, controller 110 can use trained models to quantify dosage based on a response from subject 102's brain 104 to stimulation. System 100 can implement limits on the amount of time that the system 100 can be used, monitor the cumulative dose delivered to various brain areas, enforce a maximum amount of current that can be output by emitters 120, or administer integrated dose control.

There has previously been no way to quantify the dosage of vagus nerve stimulation. Controller 110 provides a method of dosage quantification by measuring, for example, physiological responses, such as pupil dilation, to stimulation according to a particular set of parameters. Controller 110 can continuously track eye movement, pupil dilation, and other physiological responses and quantify how effective a particular set of stimulation parameters is.

In some implementations, controller 110 can quantify the effectiveness of a particular set of stimulation parameters by monitoring a differential response. For example, controller 110 can effectively “trap and trace” brain signals, such as pain signals, originating from a subject's brain. By comparing the characteristics of the brain signals, controller 110 can detect differential changes in response from a subject 102.

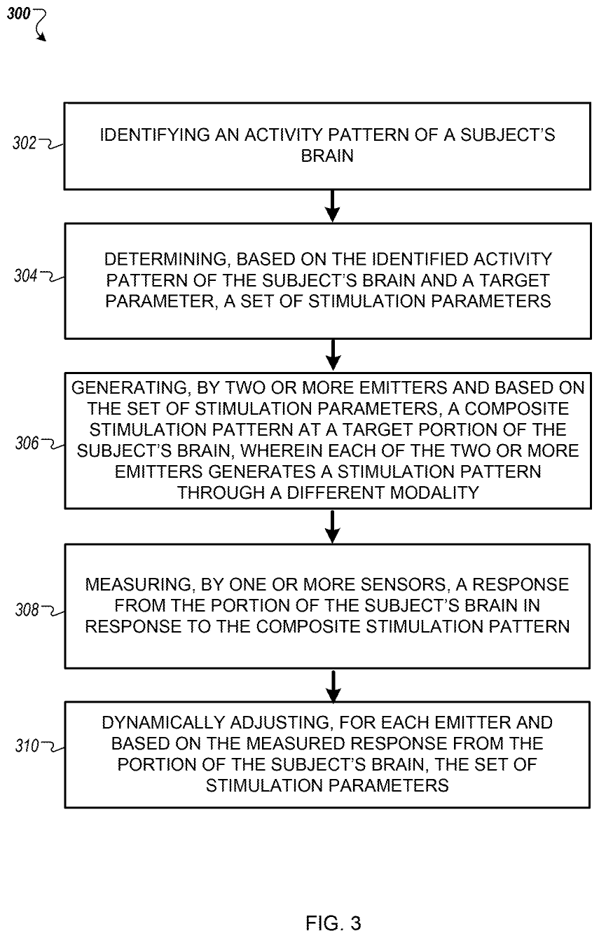

FIG. 3 is a flow chart of an example process 300 of multimodal brain stimulation. Process 300 can be implemented by multimodal brain stimulation systems such as system 100 as described above with respect to FIGS. 1 and 2. In this particular example, process 300 is described with respect to system 100 in the form of a portable headset or helmet that can be used by a subject without the supervision of a medical professional.

Briefly, according to an example, the process 300 begins with identifying an activity pattern of a subject's brain (302). For example, controller 110 can measure and identify an activity pattern of subject's brain 104. Controller 112 can identify, for example, that subject's brain 104 is in a pink noise activity pattern.

The process 300 continues with determining, based on the identified activity pattern of the subject's brain and a target parameter, a set of stimulation parameters (304). For example, controller 110 can determine, based on identifying that subject's brain 104 is in a pink noise activity pattern and a target of a hallucinogenic brain state, a set of stimulation parameters.

The target parameter can include, for example, one or more: target brain states, modalities of stimulation, target activity patterns, user inputs of waveforms, power levels of stimulation, target objects, target sizes, target compositions, durations of stimulation, particular dosages of stimulation, target quantifications of reduction in pain, and/or target percentages in reduction of tremors, among other parameters. In some implementations, the target parameter can be determined based on subject 102's verbal feedback. For example, controller 112 can process verbal feedback from subject 102 using natural language processing to determine a target parameter.

The stimulation parameters can include, for example, a power, a waveform, a shape, a pattern, a statistical parameter, a duration, a modality (e.g., ultrasound, electrical, and/or magnetic stimulation, among other modes), a frequency, a period, a target location, a target size, and/or a target composition, among other parameters.

The process 300 continues with generating, by two or more emitters and based on the set of stimulation parameters, a composite stimulation pattern at a portion of the subject's brain, wherein each of the two or more emitters generates a stimulation pattern using a different modality (306). For example, controller 110 can generate, using ultrasound stimulation system 120 and neurosensory stimulation system 140, a composite stimulation pattern at a target portion of subject's brain 104. In this particular example, controller 110 can generate a focused ultrasound beam directed at the target portion of subject's brain 104 using ultrasound stimulation system 120. Controller 110 can additionally generate pulsed light within a portion of subject 102's field of view using neurosensory stimulation system 140.

The process 300 continues with measuring, by one or more sensors, a response from the portion of the subject's brain in response to the composite stimulation pattern (308). For example, controller 110 can operate sensors 114 to measure, within a few seconds, and thus contemporaneously or near-contemporaneously with the generating step, brain activity from the target area within subject's brain 104. For example, sensors 114 can detect, using EEG, brain activity from the target area within the subject's brain 104 in response to the composite stimulation pattern.

The process 300 concludes with dynamically adjusting, for each emitter and based on the measured response from the portion of the subject's brain, the set of stimulation parameters (310). For example, controller 110 can determine, based on the measured brain activity detected by sensors 114, that subject 102 is slowly entering a target hallucinogenic brain state, but has not reached the complexity of the target state. Controller 110 can then determine, using the measured brain activity and the target brain pattern, stimulation parameters for ultrasound stimulation emitters 120. Controller 110 can also determine, using the measured brain activity and the target brain pattern, stimulation parameters for neurosensory stimulation system 140 to continue inducing the active network state in the subject's brain 104. Controller 110 can operate ultrasound stimulation system 120 and neurosensory stimulation system 140 according to the determined stimulation parameters to adjust the composite stimulation pattern. For example, controller 110 can operate ultrasound stimulation system 120 and neurosensory stimulation system 140 to alter the frequency and amplitude of the composite stimulation pattern, thus facilitating a closed loop stimulation system. Controller 110 can operate ultrasound stimulation system 120 and neurosensory stimulation system 140 with a phase shift relative to a detected in-phase large-scale brain network, enhancing or decreasing the phase lock of the brain network. Controller 110 can operate ultrasound stimulation system 120 and neurosensory stimulation system 140 with a frequency shift relative to a detected in-phase large-scale brain network, increasing or decreasing the frequency of the phase-locked brain network.

In some implementations, dynamically adjusting, for each emitter and based on the measured response from the portion of the subject's brain, a set of stimulation parameters comprises using machine learning or artificial intelligence techniques to generate one or more adjusted stimulation parameters. For example, controller 110 can apply machine learning techniques to generate adjusted stimulation parameters for one or more of ultrasound stimulation system 120, immersive virtual reality system 130, and neurosensory stimulation system 140.

In some implementations, the process includes controlling, based on the dynamically adjusted set of stimulation parameters, a set of one or more zone plates. For example, controller 110 can control an array of zone plates within system 100 to steer and/or focus the stimulation signals.

In some implementations, the process includes generating, by an immersive virtual reality system, based on the set of stimulation parameters, and for presentation to the subject, a visual representation of a scene and displaying, to the subject, the visual representation of the scene. For example, controller 110 can operate immersive virtual reality system 130 to generate a visual representation of a scene based on the target parameters and display the scene to subject 102.

A number of implementations have been described. Nevertheless, it will be understood that various modifications may be made without departing from the spirit and scope of the disclosure. For example, various forms of the flows shown above may be used, with steps re-ordered, added, or removed.

All of the functional operations described in this specification may be implemented in digital electronic circuitry, or in computer software, firmware, or hardware, including the structures disclosed in this specification and their structural equivalents, or in combinations of one or more of them. The techniques disclosed may be implemented as one or more computer program products, i.e., one or more modules of computer program instructions encoded on a computer-readable medium for execution by, or to control the operation of, data processing apparatus. The computer readable-medium may be a machine-readable storage device, a machine-readable storage substrate, a memory device, a composition of matter affecting a machine-readable propagated signal, or a combination of one or more of them. The computer-readable medium may be a non-transitory computer-readable medium. The term “data processing apparatus” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) may be written in any form of programming language, including compiled or interpreted languages, and it may be deployed in any form, including as a standalone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory may be supplemented by, or incorporated in, special purpose logic circuitry.

To provide for interaction with a user, the techniques disclosed may be implemented on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input.

Implementations may include a computing system that includes a back end component, e.g., as a data server, or that includes a middleware component, e.g., an application server, or that includes a front end component, e.g., a client computer having a graphical user interface or a Web browser through which a user may interact with an implementation of the techniques disclosed, or any combination of one or more such back end, middleware, or front end components. The components of the system may be interconnected by any form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Thus, particular implementations have been described. Other implementations are within the scope of the following claims. For example, the actions recited in the claims may be performed in a different order and still achieve desirable results.

What is claimed is:

1. A method for neurostimulation comprising:
identifying an activity pattern of a subject's brain;
determining, based on the identified activity pattern of the subject's brain and a target parameter, a set of stimulation parameters;
generating, by two or more emitters and based on the set of stimulation parameters, a composite stimulation pattern at a portion of the subject's brain, wherein each of the two or more emitters generates a stimulation pattern using a different modality;
measuring, by one or more sensors, a response from the portion of the subject's brain in response to the composite stimulation pattern; and
dynamically adjusting, for each emitter and based on the measured response form the portion of the subject's brain, a set of stimulation parameters.

2. The method of claim 1, wherein the target parameter is a selected set of one or more physiological measurements of the subject.

3. The method of claim 1, wherein the target parameter is determined based on the subject's feedback.

4. The method of claim 1, wherein the different modalities are selected from among ultrasound, pulsed light, or immersive virtual reality.

5. The method of claim 1, wherein generating, by two or more emitters and based on the set of stimulation parameters, a composite stimulation pattern at a portion of the subject's brain comprises:
generating, by a first emitter that generates a first stimulation pattern using ultrasound; and
generating, by a second emitter that generates a second stimulation pattern using pulsed light.

6. The method of claim 5, further comprising:
generating, by an immersive virtual reality system, based on the set of stimulation parameters, and for presentation to the subject, a visual representation of a scene; and
displaying, to the subject, the visual representation of the scene.

7. The method of claim 1, wherein dynamically adjusting, for each emitter and based on the measured response from the portion of the subject's brain, a set of stimulation parameters comprises using machine learning or artificial intelligence techniques to generate one or more adjusted stimulation parameters.

8. The method of claim 1, further comprising controlling, based on the dynamically adjusted set of stimulation parameters, a set of one or more zone plates.

9. A system comprising:
one or more processors; and
one or more memory elements including instructions that, when executed, cause the one or more processors to perform operations including:
identifying an activity pattern of a subject's brain;
determining, based on the identified activity pattern of the subject's brain and a target parameter, a set of stimulation parameters;
generating, by two or more emitters and based on the set of stimulation parameters, a composite stimulation pattern at a portion of the subject's brain, wherein each of the two or more emitters generates a stimulation pattern using a different modality;
measuring, by one or more sensors, a response from the portion of the subject's brain in response to the composite stimulation pattern; and
dynamically adjusting, for each emitter and based on the measured response from the portion of the subject's brain, a set of stimulation parameters.

10. The system of claim 9, wherein the target parameter is a selected set of one or more physiological measurements of the subject.

11. The system of claim 9, wherein the target parameter is determined based on the subject's feedback.

12. The system of claim 9, wherein the different modalities are selected from among ultrasound, pulsed light, or immersive virtual reality.

13. The system of claim 9, wherein generating, by two or more emitters and based on the set of stimulation parameters, a composite stimulation pattern at a portion of the subject's brain comprises:
generating, by a first emitter that generates a first stimulation pattern using ultrasound; and
generating, by a second emitter that generates a second stimulation pattern using pulsed light.

14. The system of claim 13, the operations further comprising:
generating, by an immersive virtual reality system, based on the set of stimulation parameters, and for presentation to the subject, a visual representation of a scene; and
displaying, to the subject, the visual representation of the scene.

15. The system of claim 9, wherein dynamically adjusting, for each emitter and based on the measured response from the portion of the subject's brain, a set of stimulation parameters comprises using machine learning or artificial intelligence techniques to generate one or more adjusted stimulation parameters.

16. The system of claim 9, the operations further comprising controlling, based on the dynamically adjusted set of stimulation parameters, a set of one or more zone plates.

17. A computer-readable storage device storing instructions that when executed by one or more processors cause the one or more processors to perform operations comprising:
identifying an activity pattern of a subject's brain;
determining, based on the identified activity pattern of the subject's brain and a target parameter, a set of stimulation parameters;
generating, by two or more emitters and based on the set of stimulation parameters, a composite stimulation pattern at a portion of the subject's brain, wherein each of the two or more emitters generates a stimulation pattern using a different modality;
measuring, by one or more sensors, a response from the portion of the subject's brain in response to the composite stimulation pattern; and
dynamically adjusting, for each emitter and based on the measured response form the portion of the subject's brain, a set of stimulation parameters.

18. The computer-readable storage device of claim 17, wherein the target parameter is a selected set of one or more physiological measurements of the subject.

19. The computer-readable storage device of claim 17, wherein the different modalities are selected from among ultrasound, pulsed light, or immersive virtual reality.

20. The computer-readable storage device of claim 17, wherein generating, by two or more emitters and based on the set of stimulation parameters, a composite stimulation pattern at a portion of the subject's brain comprises:
generating, by a first emitter that generates a first stimulation pattern using ultrasound; and
generating, by a second emitter that generates a second stimulation pattern using pulsed light.
